(19) United States
(12) Patent Application Publication
(10) Pub. No.: US 2004/0202732 A1
(43) Pub. Date: Oct. 14, 2004

(54) Composition to promote weight loss

(76) Inventors: William Stewart Brown, Toronto (CA); Tara Nadine Stubensey, Toronto (CA); Alan Christopher Logan, Mahwah, NJ (US); Alicja Wojewnik-Smith, Toronto (CA)

Correspondence Address: IVOR M. HUGHES, BARRISTER & SOLICITOR,, PATENT & TRADEMARK AGENTS, 175 COMMERCE VALLEY DRIVE WEST, SUITE 200, THORNHILL, ON, L3T 7P6, CA

(21) Appl. No.: 10/411,340

(22) Filed: Apr. 11, 2003

(51) Int. Cl. A61K 35/78; A61K 31/353; A61K 31/202

(52) U.S. Cl. 424/729; 514/27; 514/456; 514/560

(57) ABSTRACT

A synergistic composition of green tea and preferably an extract thereof for example epigallocatechin galate (EGCG) and linoleic acid, preferably the conjugated form, to assist with weight loss in humans.

### FIELD OF THE INVENTION

This invention relates generally to dietary supplements that stimulate weight loss, and the preferred unique composition of green tea extract and linoleic acid designed to stimulate such weight loss.

### BACKGROUND OF THE INVENTION

Each year, obesity causes at least 300,000 excess deaths in the U.S., and the healthcare costs for American adults with obesity amount to approximately $100 billion. These mortality rates have increased by 60% over the last 10 years and affect virtually all ethnic, racial and socioeconomic populations, both genders and all age groups.

Obesity, or perhaps better referred to as the “silent epidemic”, is associated with about 30 diseases or conditions including high blood pressure, diabetes (type 2), heart disease, stroke, gallbladder disease and breast, prostate and colon cancers. Three quarters of the population could suffer the ill effects of carrying excess weight within as little as ten years.

An estimated 120 million US adults are overweight or obese comparing to 800,000 affected with HIV, 9 million with cancer and 26 million with heart disease. (American Obesity Association, 2002). This excess body weight has often been wrongly attributed to genetic factors when the true cause may in fact include in our sedentary lifestyles resulting from modernization and the growth of industry and technology. We walk less, relying more on our vehicles to travel to work. Our appliances, escalators, and vacuums have all saved us countless hours of effort. When we're not on the couch, we find ourselves overwhelmed with work, discovering the convenience of the poor choices provided in fast foods. Our television sets and our computers have become our close companions leaving less time for cultivating social relationship with family and friends. In essence, today's lifestyle consists of minimal physical activity. Food is abundant, inexpensive and widely available. The food preferred by individuals are those of excessively high calories, fat and carbohydrates, and significantly low in beneficial nutrients and dietary fiber served in towering portions.

Chronic imbalance between energy intake and energy expenditure is a direct cause of obesity. This imbalance may be defined by the individuals Body Mass Index (BMI) which is a mathematical calculation used to determine whether a person is overweight/obese. BMI is calculated by dividing a person's body weight in kilograms by their height in meters squared. A BMI of 30 or greater is considered obese and a BMI between 25-29.9 is considered overweight. More than a half of Americans have a body mass index of 25 or more, which classifies them as overweight or obese.

Despite a plethora of popular diet books, commercial and educational programs on how to loose weight the results seen in society are disappointing. Even the general shift in eating patterns to low-fat foods, may simply result in people taking the same if not more total calories, usually from eating more carbohydrates. The ideal weight loss diet should create a deficit of 500 to 1000 kcal per day and be aimed at achieving a weight loss of 1 to 2 lb per week. It is known as the “balanced deficit diet”. The goal is to decrease body weight by 10 to 15% and stay at that level for 6 months to a year before starting to lose more weight.

They're only 4 ways to treat obesity:

1. Control of energy intake, mainly through modification of ones diet and/or appetite (satiety);

2. Low risk improvements in energy expenditure, essentially through the increase of thermogenesis and its effect on ones metabolism;

3. Control and reduction of fat reserves through modulation of lipogenesis (transformation of non fat food into body fat) and lipolysis (decomposition of fat) in white adipose (fat) tissue;

4. Control of availability of nutrients to cells and tissues through use of hormonal and other metabolic factors.

Many products are currently marketed to increase weight loss, but few have actually been shown to be effective in scientific studies in humans. The new weight loss and maintenance products described herein have been formulated using ingredients in amounts proven in research for their effectiveness.

Applicant is aware of a Weight Loss Kit marketed through the internet by Holista® (Holista Health (Canada) Inc.) which distributes a package of separate containers; one has Green Tea leaf in capsule form and the other container has CLA (Conjugated Linoleic Acid).

The packaging states that Green Tea Leaf from extract which is purported to have a “thermogenic” effect increasing energy expenditure and basically causing the body to burn fuel (such as fat) to create heat. The product also includes magnesium stearate, rice flour, gelatine and purified water, as well as 200 mg/capsule.

The other separate container includes 1000 mg capsules of Conjugated Linoleic Acid (CLA) from high linoleic acid, sunflower or safflower oil plus gelatine, glycerine, carob, purified water and titanium dioxide.

The products are labelled as follows:

1) Green Tea Leaf @ 200 mg @ 2 capsules×3 times/day; magnesium stearate, rice flour, gelatine, and purified water.

2) CLA @ 600 mg (from high linoleic acid flower or safflower oil 1000 mg) @ 1 capsule×3 times/day; gelatine, glycerine, carob, purified water and titanium dioxide (from naturally occurring mineral source).

The product is marketed as a Weight Loss Kit: However, there is no discussion of any synergy which might take place when using the two products. In fact there is no suggestion with the packaging of taking the products at the same time, and no discussion of the benefits of doing so.

References:

1. Obes. Rev. 2002 May; 3(2):69-74 From instinct to intellect: “The challenge of maintaining healthy weight in the modern world.” Peters J C, Wyatt H R, Donahoo W T, Hill J O.

2. American Obesity Association 2002.

3. Drugs 2002;62(6):915-44, “Pharmacological approaches for the treatment of obesity.” Fernandez-Lopez J A, Remesar X, Foz M, Alemany M.

4. Dietary Reference Intakes, Institute of Medicine of the National Academies, 2002.

5. J. Nutr. 1999 November;129(11):2037-42, “Dietary conjugated linoleic acids increase lean tissue and decrease fat deposition in growing pigs.” Ostrowska E, Muralitharan M, Cross R F, Bauman D E, Dunshea F R.

6. Int. J. Obes. Relat. Metab. Disord. 2001 August; 25(8):1129-35 “Conjugated linoleic acid (CLA) reduced abdominal adipose tissue in obese middle-aged men with signs of the metabolic syndrome: a randomised controlled trial.” Riserus U, Berglund L, Vessby B.

7. Thom E, Wadstein J, Gudmundsen O., “Conjugated linoleic acid reduces body fat in healthy exercising humans.” J Int Med Res. 2001 September-October; 29(5):392-6.

8. Mougios V, Matsakas A, Petridou A, Ring S, Sagredos A, “Melissopoulou A, Tsigilis N, Nikolaidis M. Effect of supplementation with conjugated linoleic acid on human serum lipids and body fat.” J Nutr Biochem. 2001 October; 12(10):585-594.

9. Smedman A, Vessby B., “Conjugated linoleic acid supplementation in humans—metabolic effects.” Lipids. 2001 August; 36(8):773-81.

10. Zambell K L, Horn W F, Keim N L., “Conjugated linoleic acid supplementation in humans: effects on fatty acid and glycerol kinetics.” Lipids. 2001 August; 36(8):767-72.

11. Kelly G S., “Conjugated linoleic acid: a review.” Altern Med Rev. 2001 August; 6(4):367-82. Review. PMID: 11578253.

12. Benito P, Nelson G J, Kelley D S, Bartolini G, Schmidt P C, Simon V. “The effect of conjugated linoleic acid on plasma lipoproteins and tissue fatty acid composition in humans.” Lipids 2001 March; 36(3):229-36.

13. Dyck D J., “Dietary fat intake, supplements, and weight loss.” Can J Appl Physiol. 2000 December; 25(6):495-523. Review.

14. Medina E A, Horn W F, Keim N L, Havel P J, Benito P, Kelley D S, Nelson G J, Erickson K L., “Conjugated linoleic acid supplementation in humans: effects on circulating leptin concentrations and appetite.” Lipids. 2000 July; 35(7):783-8.

15. Zambell K L, Keim N L, Van Loan M D, Gale B, Benito P, Kelley D S, Nelson G J., “Conjugated linoleic acid supplementation in humans: effects on body composition and energy expenditure.” Lipids. 2000 July; 35(7):777-82.

16. Basu S, Smedman A, Vessby B., “Conjugated linoleic acid induces lipid peroxidation in humans.” FEBS Lett. 2000 February 18; 468(1):33-6.

17. Blankson H, Stakkestad J A, Fagertun H, Thom E, Wadstein J, Gudmundsen O., “Conjugated linoleic acid reduces body fat mass in overweight and obese humans.” J Nutr. 2000 December; 130(12):2943-8.

18. Tsuboyama-Kasaoka N, Takahashi M, Tanemura K, Kim H J, Tange T, Okuyama H, Kasai M, Ikemoto S, Ezaki O., “Conjugated linoleic acid supplementation reduces adipose tissue by apoptosis and develops lipodystrophy in mice.” Diabetes 2000 September; 49(9):1534-42.

19. Azain M J, Hausman D B, Sisk M B, Flatt W P, Jewell D E., “Dietary conjugated linoleic acid reduces rat adipose tissue cell size rather than cell number.” J Nutr. 2000 June; 130(6):1548-54.

20. Bell S J, Goodrick G K., “A functional food product for the management of weight.” Crit Rev Food Sci Nutr. 2002 March; 42(2):163-78.

21. Chantre P, Lairon D. “Recent findings of green tea extract AR25 (Exolise) and its activity for the treatment of obesity.” Phytomedicine 2002 January; 9(1):3-8.

22. Dulloo A G, Seydoux J, Girardier L, Chantre P, Vandermander J., “Green tea and thermogenesis: interactions between catechin-polyphenols, caffeine and sympathetic activity.” Int J Obes Relat Metab Disord. 2000 February; 24(2):252-8.

23. Dulloo A G, Duret C, Rohrer D, Girardier L, Mensi N, Fathi M, Chantre P, Vandermander J., “Efficacy of a green tea extract rich in catechin polyphenols and caffeine in increasing 24-h energy expenditure and fat oxidation in humans.” Am J Clin Nutr. 1999 December; 70(6):1040-5.

24. Thom E, Wadstein J, Gudmundsen O., “Conjugated linoleic acid reduces body fat in healthy exercising humans.” J Int Med Res. 2001 September-October; 29(5):392-6.

25. Arner., “Differences in lipolysis between human subcutaneous and omental adipose tissues.” Ann Med 1995; 27: 435-8.

26. Richelsen, et al., “Regional differences in triglyceride breakdown in human adipose tissue: effects of catecholamines, insulin, and prostaglandin E2”, Metabolism 1991; 40: 990-6.

27. Gaskins, et al., “Evidence for abnormal prostaglandin synthesis inobese zucker rat cell.” J Nutr 1989; 119: 458-62.

28. Kavanaugh, et al., “Effect of dietary conjugated linoleic acid on phrbol ester-induced PGE2 production and hyperplasia in mouse epidermis.” Nutr Cancer 1999; 33: 132-8.

29. Katiyar, et al., “Polyphenolic antioxidant (−)-epigallocatechin-3-gallate from green tea reduces UVB-induced inflammatory responses and infiltration of leukocytes in human skin.” Photochem Photobiol 1999; 69: 148-53.

30. August, et al., “Ingestion of green tea rapidly decreases prostaglandin E2 levels in rectal mucosa in humans.” Cancer Epidemiol Biomark Prev 1999; 8: 709-13.

31. Abate, et al., “Synergistic inhibition of cyclooxgenase-2 expression by vitamin E and aspirin.” Free Radic Biol Med 2000; 29: 1135-42.

32. Bingham, “The pathogenesis of rheumatoid arthritis: pivotal cytokines involved in bone degradation and inflammation.” J Rheumatol Suppl 2002; 65: 3-9.

33. Yu, et al., “Conjugated linoleic acid decreases production of pro-inflammatory products in macrophages: evidence for a PPAR gamma-dependent mechanism.” Biochim Biophys Acta 2002; 1581: 89-99.

34. Suganuma, et al. “Mechanisms of cancer prevention by green tea polyphenols based on inhibition of TNF-alpha expression.” Biofactors 2000; 13: 67-72.

Accordingly, there is still a need for a more effective well tolerated combination product that provides safe effective weight management therapies which can be used in a broad spectrum of circumstances for individuals and without repetitive physician monitoring or costly and dangerous prescriptions or over the counter unnatural products. Ideally, the components of such a product would be formulated from reasonable cost ingredients which are not drugs and which support overall success in a weight loss program.

It is therefore a primary object of the invention to provide a composition of ingredients having a synergistic effect in addressing weight loss physiology.

It is yet a further object of the invention to provide a method of treating obesity by administering an therapeutically effective amount of a composition of ingredients having a synergistic effect in addressing weight loss physiology.

It is yet a further object of this invention to provide a composition which increases thermogenesis to promote fat burning.

It is yet a further object of the invention to control body fat reserves through modulation of lipogenesis (transformation of non fat food into body fat) and lipolysis (decomposition of fat) in white adipose (fat) tissue.

It is yet a further object of this invention to provide a composition which supports overall success in a weight loss program.

Further and other objects of this invention will become apparent to a man skilled in the art when considering the following summary of the invention and the more detailed description of the preferred embodiments described herein.

### SUMMARY OF THE INVENTION

According to a primary aspect of the invention there is provided a unique synergistic composition designed to reduce body fat mass in obese and overweight subjects combining two ingredients with proven effectiveness addressing a number of control mechanism in the reduction of body fat mass by:

1. Increasing energy expenditure;

2. Decreasing fat reserves by:

decreasing fat absorption by inhibiting the lipases (gastric and pancreatic) activity and by reduction of lipid uptake by fat cells;

promotion of lipolysis (fat cells decomposition);

3. Promoting of lean body mass.

This invention provides compositions which supports overall success in a weight loss program based upon the discovery that control of fat reserves through modulation of lipogenesis (transformation of non fat food into body fat) and lipolysis (decomposition of fat) in white adipose (fat) tissue. This is accomplished by providing a synergistic composition of green tea and preferably an extract thereof for example epigallocatechin galate (EGCG) and linoleic acid, preferably the conjugated form, to assist with weight loss in humans.

According therefore to one aspect of the invention there is provided a composition for weight loss in humans comprising a synergistic combination of effective amounts of conjugated linoleic acid (preferably in substantially in the range of about 1.7 g to about 5.1 g per day) and epigallocatechin galate (EGCG) preferably from green tea (preferably in substantially in the range of about 135 mg to about 405 mg per day) to assist with weight management. In one embodiment the composition is administered in combination with an acceptable diluent or carrier and may further be administered in aliquot dosages/amounts each day.

According to yet another aspect of the invention there is provided a composition for assisting reduction of the weight of a human, comprising a synergistic combination with effective amounts of green tea extract, preferably epigallocatechin galate (EGCG) and linoleic acid, preferably conjugated linoleic acid to assist with weight management. In one embodiment the composition is administered in combination with an effective amount of other ingredients selected from the group consisting of acceptable diluents or carrier, and is preferably administered in aliquot dosages/amounts each day.

In a preferred embodiment the composition for effective an effective weight control program, comprises a synergistic combination with effective amounts of conjugated linoleic acid (preferably in substantially in the range of about 1.7 g to about 5.1 g per day) and epigallocatechin galate (EGCG) preferably from green tea (preferably in substantially in the range of about 135 mg to about 405 mg per day) to treat obesity and specifically reduce body fat mass.

The rationale behind combining the preferred conjugated linoleic acid (CLA) and green tea extracts (specifically epigallocatechin-gallate-EGCG) is the synergistic physiological effect in human weight loss. Based on scientific research, we expect that CLA and EGCG have a greater effect on weight loss when taken together. Both CLA and EGCG have an effect on prostaglandin E2, a chemical known to prevent the breakdown of fat.²⁵⁻²⁷ There is in vitro evidence which shows that CLA can inhibit the production of prostaglandin E2²⁸ and additional evidence that green tea in general and EGCG in particular can inhibit prostaglandin E2 production.^{29,30} It is expected that the combination of CLA and EGCG will have a greater effect together than the sum of the two parts individually. Other agents, when taken together (aspirin and vitamin E for example) can enhance the effect of one another on prostaglandin E2 production.³¹ Chemicals called cytokines are responsible for prostaglandin E2 production—they include IL-1 and TNF-alpha.³² It is known that the concomitant inhibition of both IL-1 and TNF-alpha leads to additive therapeutic value in rheumatoid arthritis, a disorder where prostaglandin E2 production plays a role.³² CLA can inhibit IL-1³³ and EGCG can inhibit TNF-alpha,³⁴ so it is postulated that the inhibition of prostaglandin E2 production by separately decreasing these cytokines (IL-1 and TNF-alpha) will lead to an enhanced effect on weight loss.

According to yet another aspect of the invention there is provided for controlling the weight of a human a synergistic composition comprising: 1) green tea or an extract or derivative thereof or the like, for example epigallocatechin-gallate; and 2) linoleic acid and preferably the conjugated form thereof or the like, which together synergistically increase thermogenesis and inhibit production of prostaglandin E2 and thereby control the weight of a human. Said composition may further comprise carriers, and excipients such as beeswax and lecithin or the like, and may further comprising sweetening agents, flavouring agents, colouring agents and/or preserving agents in order to provide palatable preparations. In a preferred embodiment the composition for effective an effective weight control program, comprises the synergistic combination with effective amounts of conjugated linoleic acid (preferably in substantially in the range of about 1.7 g to about 5.1 g per day) and epigallocatechin galate (EGCG) preferably from green tea (preferably in substantially in the range of about 135 mg to about 405 mg per day) to treat obesity and specifically reduce body fat mass.

According to yet another aspect of the invention there is provided a method for controlling the weight of a human comprising administering to said human a therapeutically effective amount, of preferably a synergistic composition of: 1) green tea or an extract or derivative thereof or the like, for example epigallocatechin-gallate; and 2) linoleic acid and preferably the conjugated form thereof or the like, which together synergistically increase thermogenesis and inhibit production of prostaglandin E2 and thereby control the weight of a human. In a preferred embodiment the method for an effective weight control program, comprises administering the synergistic combination with effective amounts of conjugated linoleic acid (preferably in substantially in the range of about 1.7 g to about 5.1 g per day) and epigallocatechin galate (EGCG) preferably from green tea (preferably in substantially in the range of about 135 mg to about 405 mg per day) to treat obesity and specifically reduce body fat mass.

### DETAILED DESRIPTION OF THE INVENTION

A synergistic composition of CLA and EGCG is provided with very strong evidence for its effectiveness in weigh loss. The suggested daily dose has been proven in number of scientific studies in humans. When provided in gelatine capsule a daily dose of 6 capsules divided into three servings at breakfast, lunch and dinner provides 3.4 g of pure CLA and 270 mg of EGCG. In one embodiment the capsules may contain gelatine, glycerine, carob and water.

This composition offers synergistic activity addressing two of the major control mechanisms in the treatment of obesity and specifically body fat mass reduction:

Increase of energy expenditure.

Decrease of fat reserves by:

decrease of fat absorption;

inhibiting lipases (fat digesting enzymes) activity;

reducing lipid uptake by fat cells and decreasing their volume;

promotion of lipolysis (fat cells decomposition).

Promotion of lean body mass.

Each capsule contains:

| | |
|---|---|
| Conjugated Linoleic acid | 567 mg |
| (From CLA oil 80%) | |
| EGCG catechins | 45 mg |
| (From Green tea extract std. to 50%) | |

It is suggested for weight maintenance one capsule be taken with each meal and

for weight loss two capsules be taken with each meal. In one embodiment the capsules may contain gelatine, glycerine, carob and water.

Typical daily doses therefore provides 3,400 mg of pure 100% CLA and 270 mg of EGCG catechins.

Conjugated Linoleic Acid (CLA) refers to a group of isomers of the omega-6 essential fatty acid—linoleic acid. The primary dietary sources of CLA are animal products (average CLA content in diary products range from 3 to 9 mg/g fat). Vegetable fats are generally poorer sources of CLA. However, CLA is produced from linoleic acid in safflower and sunflower oil by special treatment. CLA been shown to play a role in the inhibition of tumor cell growth, in the inhibition of diabetes and atherosclerosis (deposits of cholesterol in the arteries) as well as in the alteration of body composition.

The mixed isomers of CLA, mainly consisting of equal amounts of the cis 9 trans 11 and trans 10 cis 12 isomers, seem to be responsible for the induction of changes in body composition. People either form more adipocytes (fat cells) and/or existing adipocytes absorb too much fat-glucose and become larger. “CLA makes big fat cell get little and stay that way”.

A large number of studies indicate that CLA reduces lipid uptake by the fat cell thus decrease the volume of adipocytes. CLA has been also shown to increase fat cell decomposition (lipolysis). In addition to body fat reduction, dietary CLA increases whole body protein accumulation and in turn enhances the lean body mass. Simply CLA consistently shows an ability to reduce body fat while maintaining lean muscle mass.

The first study to directly examine the effects of CLA on body composition was by Park (1997). Mice that were fed CLA supplemented diets for 28 days maintained body mass but significantly reduced body fat by up to 60% and increased body protein up to 13%.

In a particularly significant study published in the International Journal of Obesity, CLA has been shown to be effective in decreasing abdominal fat.

This double blind randomized placebo controlled trial observed twenty-five men with significant abdominal fat for four weeks while supplemented with CLA. The 4-week treatment induced a significant reduction in sagittal abdominal diameter (SAD)—an average of one-inch reduction from their waistlines, without any cardiovascular risk factors. Similar study showing effectiveness of CLA in reducing the abdominal obesity has been published in the Clinical Science.

In the study published in Lipids 2001 fifty-three healthy men and women were randomly assigned to supplementation with CLA for 12 weeks. At the end of the study the proportion of body fat decreased significantly within the CLA-treated group by 3.8%.

In another randomized, double blind, placebo controlled study sixty overweight or obese subjects were treated with CLA over 12 weeks. The daily dosage was divided into three doses taken at breakfast, lunch and dinner. A significant reduction in body fat mass (BFM) was found in the 3.4-g and higher CLA groups.

CLA has been also shown to reduce body fat mass in healthy exercising humans. Participants in this study—healthy humans of normal body weight and body mass index of less than 25, did standardized physical exercise in a gym for 90 minutes three times weekly. They have been observed taking CLA in three daily doses for 2 weeks. Body fat was significantly reduced in the CLA group versus placebo wherein no effects on body weight loss were observed.

While many published studies document the fat reducing effects of CLA, the fact that CLA may also protect against cancer, vascular disease and type II diabetes without any side effects makes it a preferred supplement to use daily. CLA is very well tolerated and no side effects or drug interactions have been reported.

Energy homeostasis is regulated through food intake (hunger and satiety), energy expenditure and the secretion of hormones that regulate use and storage of nutrients. Energy expenditure is regulated by the resting metabolic rate (RMR), physical activity and dietary thermogenesis.

Thermogenesis refers to the body's production of heat, a normal part of metabolic process. Thermogenesis is also a mechanism that increases metabolic rate. Stored body fat, if released and available for use, can provide the fuel for this increased metabolic rate. Thermogenesis can be enhanced by number of nutritional substances.

Green tea has been shown to induce thermogenesis.

It is one of the ancient beverages made from the leaves of Camelia sinensis. As early as 2737 B.C., the Chinese Emperor Shen Nung discovered tea and hailed it as a miracle of natural medicine. This popular beverage has been shown to, lower cholesterol and blood pressure, protect against certain cancers, block bacteria and viruses, improve digestion, and reduce the risk of ulcers and strokes. Green tea is the non-oxidized, nonfermented product and contains high quantities of several polyphenolics components such as epicatechin and epigallocatechin galate (EGCG). EGCG found in green tea cannot be obtained in appreciable amounts from any other food sources. Black tea leaves have a much lower content of these catechins. That's because black tea leaves undergo extensive fermentation, during which the majority of catechins are enzymatically oxidized. Green tea by containing high quantities of pharmacologically active catechins and caffeine exert an antiobesity action by inhibition of lipases as well as promotion of thermogenesis.

Fat digestion starts in the stomach where gastric lipase (fat digesting enzyme) hydrolyzes some of dietary fat under acidic conditions. In the small intestine lipolysis is completed by pancreatic lipase with other cofactors and the absorption of lipolytic products starts.

Research shows that green tea extract with high content of EGCG has an ability to reduce gastric and intestinal fat digestion by inhibiting activity of lipases and well as reducing lipid emulsification process. Many in vitro and animal studies have shown the antiobesity activity of green tea.

In the recent human studies on activity of green tea in moderately obese patients, body weight was decreased by 4.6% and waist circumference by 4.5% after 3 months. There have been a significant increase in energy expenditure (4%) without any increase in heart rate. This side effect of many antiobesity thermogenic agents such as caffeine limits their use by obese patients because of hypertension and other cardiovascular complications.

270 mg of EGCG divided into three doses has been used in the studies. The mechanism of action of the increased resting energy expenditure, and the fat oxidation must be independent of caffeine because these changes did not occur with caffeine administration alone.

Typically, thermogenesis contributes 0-8 0 % of the daily energy expenditure, and the addition of green tea increases that figure to 35 to 43% (0-75 kcal). Although seemingly small for a single day, the long-term effects are significant. Caloric expenditure of 75 kcal per day theoretically may results in 8 lb of weight loss per year, and this is in addition to lipase inhibition, which also promotes weight loss. It has been shown in vitro that green tea extract inhibits gastric and pancreatic lipases by 37%.

In addition to a strong scientific evidence of anticancer and other benefits of green tea, this supplement is a very effective weight control agent. Ability of inhibiting lipase enzymes and reducing fat break down in our body and into our bloodstream mimics weight control drug Xenical without any side effects like uncontrolled diarrhea.

Although the description above contains many specifics, these should not be construed as limiting the scope of the invention but as merely providing illustrations of some of the presently preferred embodiments of this invention. Various other embodiments and ramifications are possible within it's scope.

As many changes can be made to the preferred embodiments of the invention without departing from the scope thereof. It is intended that all matter contained herein be considered illustrative of the invention and not it a limiting sense.

1. A composition comprising an orally administered synergistic weight loss combination of epigallocatechin gallate(EGCG) and conjugated linoleic acid, wherein said composition assists with weight loss in humans.

2. A composition for weight loss in humans comprising an orally administered synergistic weight loss combination of effective amounts of conjugated linoleic acid substantially in the range of 1.7 g to about 5.1 g per day) and epigallocatechin gallate (EGCG) substantially in the range of 135 mg to about 405 mg per day to assist with weight management.

3. A composition for assisting reduction of body weight comprising an orally administered synergistic weight loss combination with effective amounts of the green tea extract epigallocatechin gallate (EGCG) and conjugated linoleic acid to assist with weight management.

4. The composition of claim 1, or 3 wherein effective amounts of conjugated linoleic acid administered is substantially in the range of 1.7 g to 5.1 g per day.

5. The composition of claim 1, or 3 wherein effective amounts of epigallocatechin gallate (EGCG) from green tea consumed is substantially in the range of 135 mg to 405 mg per day.

6. The composition of claim 4 wherein effective amounts of epigallocatechin gallate (EGCG) from green tea consumed is substantially in the range of 135 mg to 405 mg per day.

7. The composition of claim 1, 2 or 3 administered in combination with an acceptable diluent or carrier.

8. The composition of claim 1, 2 or 3 administered in aliquot dosages/amounts each day.

9. For controlling body weight of a human, a composition comprising an orally administered synergistic weight loss combination of effective amounts of 1) epigallocatechin-gallate and 2) conjugated linoleic acid which together synergistically effectively increase thermogenesis and inhibit production of prostaglandin E2 and thereby control human body fat reserves through modulation of lipogenesis (transformation of non fat food into body fat) and lipolysis (decomposition of fat) in white adipose (fat) tissue.

10. The composition of claim 9 further comprising effective amounts of conjugated linoleic acid substantially in the range of 1.7 g to 5.1 g per day and epigallocatechin gallate (EGCG) substantially in the range of 135 mg to 405 mg per day to treat obesity and specifically reduce body fat mass.

11. A method for controlling body weight of a human comprising administering to said human a therapeutically effective amount, of a composition comprising an orally administered synergistic weight loss combination of effective amounts of 1) epigallocatechin-gallate and 2)conjugated linoleic acid which together synergistically increase thermogenesis and inhibit production of prostaglandin E2 and thereby modulate lipogenesis (transformation of non fat food into body fat) and lipolysis (decomposition of fat) in white adipose (fat) tissue to thereby control the weight of a human.

12. The method of claim 11 further comprising administering to said human effective amounts of conjugated linoleic acid substantially in the range of 1.7 g to 5.1 g per day and epigallocatechin gallate (EGCG) substantially in the range of 135 mg to 405 mg per day) to treat obesity and specifically reduce body fat mass.

13. The compositions of any preceeding claim further comprising sweetening agents, flavouring agents, colouring agents and/or preserving agents in order to provide palatable preparations.
